(19) United States
(12) Patent Application Publication
(10) Pub. No.: US 2010/0317880 A1
(43) Pub. Date: Dec. 16, 2010

(54) Direct epoxidation process using modifiers

(76) Inventors: Roger A. Grey, West Chester, PA (US); Andrew P. Kahn, Eagleville, PA (US)

Correspondence Address: LyondellBasell Industries, 3801 WEST CHESTER PIKE, NEWTOWN SQUARE, PA, 19073, US

(21) Appl. No.: 12/456,090

(22) Filed: Jun. 11, 2009

(51) Int. Cl. C07D 301/06

(52) U.S. Cl. 549/533

(57) ABSTRACT

The invention is a process for epoxidizing an olefin with hydrogen and oxygen in a solvent comprising tertiary butyl alcohol or acetonitrile in the presence of an amide modifier and a catalyst comprising titanium-MWW zeolite and a noble metal. The process produces less ring-opened products such as glycols and glycol ethers when performed in the presence of the amide, while maintaining low alkane byproduct formed by the hydrogenation of olefin.

### FIELD OF THE INVENTION

This invention relates to a process for producing an epoxide by the reaction of an olefin, oxygen, and hydrogen.

### BACKGROUND OF THE INVENTION

Many different methods for the preparation of epoxides have been developed. Generally, epoxides are formed by the reaction of an olefin with an oxidizing agent in the presence of a catalyst. Ethylene oxide is commercially produced by the reaction of ethylene with oxygen over a silver catalyst. Propylene oxide is commercially produced by reacting propylene with an organic hydroperoxide oxidizing agent, such as ethylbenzene hydroperoxide or tert-butyl hydroperoxide. This process is performed in the presence of a solubilized molybdenum catalyst, see U.S. Pat. No. 3,351,635, or a heterogeneous titania on silica catalyst, see U.S. Pat. No. 4,367,342.

Besides oxygen and alkyl hydroperoxides, hydrogen peroxide is also a useful oxidizing agent for epoxide formation. U.S. Pat. Nos. 4,833,260, 4,859,785, and 4,937,216, for example, disclose olefin epoxidation with hydrogen peroxide in the presence of a titanium silicate catalyst. Cheng, et al., J. Catal. 255 (2008) 343, describe the epoxidation of propylene over a TS-1 catalyst using urea plus hydrogen peroxide as oxidizing agent. U.S. Pat. Nos. 7,153,986 and 7,531,674 describe the epoxidation of propylene with hydrogen peroxide in the presence of an organic solvent and a crystalline titanosilicate catalyst having an MWW structure.

Much current research is conducted in the direct epoxidation of olefins with oxygen and hydrogen. In this process, it is believed that oxygen and hydrogen react in situ to form an oxidizing agent. Many different catalysts have been proposed for use in the direct epoxidation process. Typically, the catalyst comprises a noble metal and a titanosilicate. For example, JP 4-352771 discloses the formation of propylene oxide from propylene, oxygen, and hydrogen using a catalyst containing a Group VIII metal such as palladium on a crystalline titanosilicate. The Group VIII metal is believed to promote the reaction of oxygen and hydrogen to form a hydrogen peroxide in situ oxidizing agent. U.S. Pat. No. 6,498,259 describes a catalyst mixture of a titanium zeolite and a supported palladium complex, where palladium is supported on carbon, silica, silica-alumina, titania, zirconia, and niobia. Other direct epoxidation catalyst examples include gold supported on titanosilicates, see for example PCT Intl. Appl. WO 98/00413.

One disadvantage of the described direct epoxidation catalysts is that they are prone to produce non-selective byproducts such as glycols or glycol ethers formed by the ring-opening of the epoxide product or alkane byproduct formed by the hydrogenation of olefin. U.S. Pat. No. 6,008,388 teaches that the selectivity for the direct olefin epoxidation process is enhanced by the addition of a nitrogen compound such as ammonium hydroxide to the reaction mixture. U.S. Pat. No. 6,399,794 teaches the use of ammonium bicarbonate modifiers to decrease the production of ring-opened byproducts.

U.S. Pat. No. 6,005,123 teaches the use of phosphorus, sulfur, selenium or arsenic modifiers such as triphenylphosphine or benzothiophene to decrease the formation of unwanted propane. U.S. Pat. No. 7,026,492 discloses that the presence of carbon monoxide, methylacetylene, and/or propadiene modifier gives significantly reduced alkane byproduct. In addition, co-pending U.S. patent application Ser. No. 11/489,086 discloses that the use of a lead-modified palladium-containing titanium or vanadium zeolite reduces alkane byproduct formation.

As with any chemical process, it is desirable to attain still further improvements in the epoxidation methods. We have discovered a new process for the epoxidation of olefins.

### SUMMARY OF THE INVENTION

The invention is an olefin epoxidation process that comprises reacting an olefin, oxygen, and hydrogen in a solvent comprising tertiary butyl alcohol or acetonitrile in the presence of an amide modifier and a catalyst comprising a titanium-MWW zeolite and a noble metal. This process surprisingly gives much lower amounts of undesired glycol and glycol ether by-products, while maintaining low alkane by-product formation by the hydrogenation of olefin, compared to processes that do not use the modifier or use a different titanium zeolite.

### DETAILED DESCRIPTION OF THE INVENTION

The process of the invention comprises reacting an olefin, oxygen, and hydrogen in the presence of a catalyst. The catalyst useful in the process of the invention comprises a titanium-MWW zeolite and a noble metal. Titanium zeolites comprise the class of zeolitic substances wherein titanium atoms are substituted for a portion of the silicon atoms in the lattice framework of a molecular sieve. Ti-MWW zeolite is a porous molecular sieve zeolite having an MEL topology analogous to that of the MWW aluminosilicate zeolites, containing titanium atoms substituted in the framework. Such substances, and their production, are well known in the art. See for example, U.S. Pat. No. 6,759,540 and Wu et al., J. Phys. Chem. B, 2001, 105, p. 2897.

The titanium-MWW zeolite preferably contain no elements other than titanium, silicon, and oxygen in the lattice framework, although minor amounts of boron, iron, aluminum, sodium, potassium, copper and the like may be present.

The titanium-MWW zeolite will generally have a composition corresponding to the following empirical formula xTiO₂ (1-x)SiO₂ where x is between 0.0001 and 0.5000. More preferably, the value of x is from 0.01 to 0.125. The molar ratio of Si:Ti in the lattice framework of the zeolite is advantageously from 9.5:1 to 99:1 (most preferably from 9.5:1 to 60:1). The use of relatively titanium-rich zeolites may also be desirable.

The catalyst employed in the process of the invention also comprises a noble metal. The noble metal is preferably incorporated into the catalyst by supporting the noble metal on the titanium-MWW zeolite to form a noble metal-containing titanium-MWW zeolite, or alternatively, the noble metal may be first supported on a carrier such as an inorganic oxide, clay, carbon, or organic polymer resins, or the like, and then physically mixed with the titanium-MWW zeolite. There are no particular restrictions regarding the choice of noble metal compound used as the source of the noble metal. For example, suitable compounds include the nitrates, sulfates, halides (e.g., chlorides, bromides), carboxylates (e.g. acetate), and amine complexes of noble metals.

A preferred catalyst useful in the process of the invention is a noble metal-containing titanium-MWW zeolite. Such catalysts typically comprise a noble metal (such as palladium, gold, platinum, silver, iridium, ruthenium, osmium, or combinations thereof) supported on a titanium-MWW zeolite. Noble metal-containing titanium zeolites are well known in the art and are described, for example, in JP 4-352771 and U.S. Pat. Nos. 5,859,265 and 6,555,493, the teachings of which are incorporated herein by reference in their entirety. The noble metal-containing titanium-MWW zeolites may contain a mixture of noble metals. Preferred noble metal-containing titanium-MWW zeolites comprise palladium and a titanium-MWW zeolite; palladium, gold, and a titanium-MWW zeolite; or palladium, platinum, and titanium-MWW zeolite.

The typical amount of noble metal present in the noble metal-containing titanium-MWW zeolite will be in the range of from about 0.001 to 20 weight percent, preferably 0.005 to 10 weight percent, and particularly 0.01 to 5 weight percent.

Another preferred catalyst useful in the process of the invention is a catalyst mixture comprising a titanium-MWW zeolite and a supported noble metal catalyst. The supported noble metal catalyst comprises a noble metal and a carrier. The carrier is preferably a porous material. Carriers are well-known in the art. For instance, the carrier can be inorganic oxides, clays, carbon, and organic polymer resins. Preferred inorganic oxides include oxides of Group 2, 3, 4, 5, 6, 13, or 14 elements. Particularly preferred inorganic oxide carriers include silica, alumina, silica-aluminas, titania, zirconia, niobium oxides, tantalum oxides, molybdenum oxides, tungsten oxides, amorphous titania-silica, amorphous zirconia-silica, amorphous niobia-silica, and the like. The carrier may be a zeolite, but is not a titanium-MWW zeolite. Preferred organic polymer resins include polystyrene, styrene-divinylbenzene copolymers, crosslinked polyethyleneimines, and polybenzimidizole. Suitable carriers also include organic polymer resins grafted onto inorganic oxide carriers, such as polyethylenimine-silica. Preferred carriers also include carbon. Particularly preferred carriers include carbon, titania, zirconia, niobia, silica, alumina, silica-alumina, tantalum oxide, molybdenum oxide, tungsten oxide, titania-silica, zirconia-silica, niobia-silica, and mixtures thereof.

Preferably, the carrier has a surface area in the range of about 1 to about 700 m²/g, most preferably from about 10 to about 500 m²/g. Preferably, the pore volume of the carrier is in the range of about 0.1 to about 4.0 mL/g, more preferably from about 0.5 to about 3.5 mL/g, and most preferably from about 0.8 to about 3.0 mL/g. Preferably, the average particle size of the carrier is in the range of about 0.1 μm to about 0.5 inch, more preferably from about 1 μm to about 0.25 inch, and most preferably from about 10 μm to about 1/16 inch. The preferred particle size is dependent upon the type of reactor that is used, for example, larger particle sizes are preferred for a fixed bed reaction. The average pore diameter is typically in the range of about 10 to about 1000 Å, preferably about 20 to about 500 Å, and most preferably about 50 to about 350 Å.

The supported noble metal catalyst also contains a noble metal. While any of the noble metals can be utilized (i.e., gold, silver, platinum, palladium, iridium, ruthenium, osmium), either alone or in combination, palladium, platinum, gold, a palladium/platinum, or a palladium/gold combination are particularly desirable. Palladium is most preferred.

Typically, the amount of noble metal present in the supported catalyst will be in the range of from 0.01 to 10 weight percent, preferably 0.01 to 4 weight percent. There are no particular restrictions regarding the choice of noble metal compound or complex used as the source of noble metal in the supported catalyst. For example, suitable compounds include the nitrates, sulfates, halides (e.g., chlorides, bromides), carboxylates (e.g. acetate), oxides, and amine complexes of the noble metal.

The catalyst useful in the process of the invention preferably contains lead, bismuth, or rhenium. The catalyst of the invention most preferably contains lead. As with the noble metal, lead, bismuth, or rhenium may be supported on the titanium-MWW zeolite or, alternatively, the lead, bismuth, or rhenium may be first supported on a carrier then physically mixed with the titanium-MWW zeolite.

Preferably, the catalyst will contain from about 0.001 to 5 weight percent of lead, bismuth, or rhenium and 0.01 to 10 weight percent of the noble metal. Most preferably, the catalyst contains 0.01 to 2 weight percent of lead, bismuth, and rhenium. Preferably, the weight ratio of noble metal to lead (bismuth or rhenium) in the catalyst is in the range of 0.1 to 10. While the choice of lead, bismuth, or rhenium compound used as the lead, bismuth, or rhenium source in the supported catalyst is not critical, suitable compounds include carboxylates (e.g., acetate, citrate), halides (e.g., chlorides, bromides, iodides), oxyhalides (e.g., oxychloride), carbonates, nitrates, phosphates, oxides, and sulfides. If used, the lead, bismuth, or rhenium may be added to the titanium-MWW zeolite or carrier before, during, or after noble metal addition.

Any suitable method may be used for the incorporation of the noble metal and optional lead, bismuth, or rhenium into the catalyst. For example, the noble metal and optional lead, bismuth, or rhenium may be supported on the titanium-MWW zeolite or the carrier by impregnation, ion-exchange, or incipient wetness techniques with, for example, palladium tetraammine chloride. If lead, bismuth, or rhenium is used, the order of addition of noble metal and optional lead, bismuth, or rhenium to the titanium-MWW zeolite or the carrier is not considered critical. However, it is preferred to add the lead, bismuth, or rhenium compound at the same time that the noble metal is introduced.

After noble metal and optional lead, bismuth, or rhenium incorporation, the noble metal-containing titanium-MWW or supported noble metal catalyst is recovered. Suitable catalyst recovery methods include filtration and washing, rotary evaporation and the like. The catalyst is typically dried prior to use in epoxidation. The drying temperature is preferably from about 50° C. to about 200° C. The catalyst may additionally comprise a binder or the like and may be molded, spray dried, shaped or extruded into any desired form prior to use in epoxidation.

After noble metal-containing titanium-MWW or supported noble metal catalyst formation, the catalyst may be optionally thermally treated in a gas such as nitrogen, helium, vacuum, hydrogen, oxygen, air, or the like. The thermal treatment temperature is typically from about 20° C. to about 800° C. It is preferred to thermally treat the catalyst in the presence of an oxygen-containing gas at a temperature from about 200° C. to 700° C., and optionally reduce the catalyst in the presence of a hydrogen-containing gas at a temperature from about 20° C. to 600° C.

In the epoxidation process of the invention, the catalyst may be used as a powder or as a large particle size solid. If a noble metal-containing titanium-MWW zeolite is used, the noble metal-containing zeolite may be used as a powder but is preferably spray dried, pelletized or extruded prior to use in epoxidation. If spray dried, pelletized or extruded, the noble metal-containing titanium-MWW zeolite may additionally comprise a binder or the like and may be molded, spray dried, shaped or extruded into any desired form prior to use in epoxidation. The noble metal-containing titanium-MWW zeolite may also be encapsulated in polymer as described in U.S. Pat. No. 7,030,255, the teachings of which are incorporated herein by reference in their entirety. If a catalyst mixture of titanium-MWW zeolite and supported noble metal catalyst is used, the titanium-MWW zeolite and supported catalyst may be pelletized or extruded together prior to use in epoxidation. If pelletized or extruded together, the catalyst mixture may additionally comprise a binder or the like and may be molded, spray dried, shaped or extruded into any desired form prior to use in epoxidation. The catalyst mixture may also be encapsulated in polymer as described in U.S. Pat. No. 7,030,255.

The epoxidation process of the invention comprises contacting an olefin, oxygen, and hydrogen in a solvent comprising tertiary butyl alcohol or acetonitrile in the presence of the amide modifier and the catalyst. Suitable olefins include any olefin having at least one carbon-carbon double bond, and generally from 2 to 60 carbon atoms. Preferably the olefin is an acyclic alkene of from 2 to 30 carbon atoms; the process of the invention is particularly suitable for epoxidizing C₂-C₆ olefins. More than one double bond may be present, as in a diene or triene for example. The olefin may be a hydrocarbon (i.e., contain only carbon and hydrogen atoms) or may contain functional groups such as halide, carboxyl, hydroxyl, ether, carbonyl, cyano, or nitro groups, or the like. The process of the invention is especially useful for converting propylene to propylene oxide.

Oxygen and hydrogen are also required for the epoxidation process. Although any sources of oxygen and hydrogen are suitable, molecular oxygen and molecular hydrogen are preferred.

The epoxidation process of the invention is carried out in the liquid (or supercritical or subcritical) phase in the presence of a solvent comprising tertiary butyl alcohol or acetonitrile. The solvent may preferably comprise co-solvents such as water, liquid CO₂, and oxygenated hydrocarbons such as alcohols, ethers, esters, ketones, and the like. It is particularly preferable to use a mixture of tertiary butyl alcohol and water.

The epoxidation process of the invention also employs one or more amide modifiers. An amide modifier is any compound that contains at least one amide functionality. Preferred amide modifiers include urea, substituted urea (e.g., R₁R₂NCONH₂), formamide, dimethyl formamide, acetamide, and carbamates (methyl, ethyl, phenyl, etc.). Particularly preferred amide modifiers include urea, formamide, and acetamide.

The amide modifier will typically be added to the reaction mixture along with the solvent. The amount of amide modifier in the reaction mixture is preferably in the range of from 0.002 molar to 1 molar, and most preferably from about 0.02 molar to 0.2 molar.

Epoxidation according to the invention is carried out at a temperature effective to achieve the desired olefin epoxidation, preferably at temperatures in the range of 0-250° C., more preferably, 20-100° C. The molar ratio of hydrogen to oxygen can usually be varied in the range of H₂:O₂=1:10 to 5:1 and is especially favorable at 1:5 to 2:1. The molar ratio of oxygen to olefin is usually 2:1 to 1:20, and preferably 1:1 to 1:10. A carrier gas may also be used in the epoxidation process. As the carrier gas, any desired inert gas can be used. The molar ratio of olefin to carrier gas is then usually in the range of 100:1 to 1:10 and especially 20:1 to 1:10.

As the inert gas carrier, noble gases such as helium, neon, and argon are suitable in addition to nitrogen and carbon dioxide. Saturated hydrocarbons with 1-8, especially 1-6, and preferably with 1-4 carbon atoms, e.g., methane, ethane, propane, and n-butane, are also suitable. Nitrogen and saturated C₁-C₄ hydrocarbons are the preferred inert carrier gases. Mixtures of the listed inert carrier gases can also be used.

Specifically in the epoxidation of propylene, propane can be supplied in such a way that, in the presence of an appropriate excess of carrier gas, the explosive limits of mixtures of propylene, propane, hydrogen, and oxygen are safely avoided and thus no explosive mixture can form in the reactor or in the feed and discharge lines.

The process may be performed using a continuous flow, semi-batch or batch mode of operation. The catalyst mixture is preferably in the form of a suspension or fixed-bed.

The following examples merely illustrate the invention. Those skilled in the art will recognize many variations that are within the spirit of the invention and scope of the claims.

### EXAMPLE 1

### Catalyst Preparation

TS-1 can be made according to any known literature procedure. See, for example, U.S. Pat. No. 4,410,501, DiRenzo, et. al., Microporous Materials (1997), Vol. 10, 283, or Edler, et. al., J. Chem. Soc., Chem. Comm. (1995), 155. Ti-MWW can be made according to Wu et al., J. Phys. Chem. B, 2001, 105, p. 2897.

Catalyst 1 (Pd—Pb/TiO₂): Lead nitrate (1.92 g) is added to a solution of deionized water (60 mL) and 30 mL of 2.56 molar nitric acid to form a lead nitrate solution, and an aqueous solution of palladium dinitrate (6.4 g, 20.64 wt. % Pd) is added with mixing. The Pd—Pb solution is then added by incipient wetness to spray dried titania (120 g, 30 micron size, 40 m²/g, calcined in air at 700° C.). The solids are calcined in air in a muffle furnace by heating at 110° C. for 4 hours (after ramping at 10° C./min) and then at 300° C. for 4 hours (after ramping at 2° C./min). The solids (80 g) are washed with aqueous sodium bicarbonate (3.6 g of NaHCO₃ in 160 g deionized water), again with aqueous sodium bicarbonate (3.6 g of NaHCO₃ in 100 g deionized water), and finally washed three times with deionized water (3×160 g), before the solids are vacuum dried at 50° C. for 18 hours. The solids are then calcined in a muffle furnace by heating at 110° C. for 4 hours (after ramping at 10° C./min) and then heating at 600° C. for 4 hours (after ramping at 2° C./min). The solids are transferred to a quartz tube and reduced with a 4 vol. % hydrogen in nitrogen stream at 100° C. for 1 hour (100 cc/hr), followed by nitrogen for 30 minutes while cooling from 100° C. to 30° C. to produce Catalyst 1. Catalyst 1 contains 0.95 wt. % Pd, 0.8 wt. % Pb, and 51 wt. % Ti.

Catalyst 2 (Pd—Pb/TiO₂): Lead nitrate (2.56 g) is added to 53 mL of 2.56 molar nitric acid to form a lead nitrate solution, and an aqueous solution of palladium dinitrate (7.75 g, 20.64 wt. % Pd) is added with mixing. The Pd—Pb solution is then added by incipient wetness to spray dried titania (80 g, 30 micron size, 40 m²/g, calcined in air at 700° C.). The solids are calcined in air in a muffle furnace by heating at 110° C. for 4 hours (after ramping at 10° C./min) and then at 300° C. for 4 hours (after ramping at 2° C./min). The solids are calcined again in a muffle furnace by heating at 110° C. for 4 hours (after ramping at 10° C./min) and then heating at 600° C. for 4 hours (after ramping at 2° C./min). The solids are then transferred to a quartz tube and reduced with a 4 vol. % hydrogen in nitrogen stream at 100° C. for 1 hour (100 cc/hr), followed by nitrogen for 30 minutes while cooling from 100° C. to 30° C. to produce Catalyst 2. Catalyst 2 contains 1.5 wt. % Pd, 1.4 wt. % Pb, and 51 wt. % Ti.

Catalyst 3 (Pd—Pb/Ti-MWW): Lead nitrate (0.13 g) is added to deionized water (14 mL) and the Pb solution is then added by incipient wetness to Ti-MWW (8 g, 10 micron size, 300 m²/g, calcined in air at 530° C.). The solids are calcined in air in a muffle furnace by heating at 110° C. for 4 hours (after ramping at 10° C./min) and then at 600° C. for 4 hours (after ramping at 2° C./min). The solids contain 0.75 wt. % Pb and 1.3 wt. % Ti. The Pb/Ti-MWW solids (4 g) are treated by incipient wetness with deionized water (8 g) containing palladium dinitrate (0.043 g aqueous solution containing 20.64 wt. % Pd). The solids are calcined in air in a muffle furnace by heating at 110° C. for 4 hours (after ramping at 10° C./min) and then at 300° C. for 4 hours (after ramping at 2° C./min). The solids are calcined again in a muffle furnace by heating at 110° C. for 4 hours (after ramping at 10° C./min) and then heated at 600° C. for 4 hours (after ramping at 2° C./min). The solids are transferred to a quartz tube and reduced with a 4 vol. % hydrogen in nitrogen stream at 100° C. for 1 hour (100 cc/hr), followed by nitrogen for 30 minutes while cooling from 100° C. to 30° C. to produce Catalyst 3. Catalyst 3 contains 0.31 wt. % Pd, 0.65 wt. % Pb, and 1.4 wt. % Ti.

### EXAMPLE 2

### Propylene Epoxidation

To evaluate the performance of the catalysts [(a) Catalyst 3 and (b) catalyst mixtures of supported Catalysts 1 or 2 with Ti-MWW or TS-1], the epoxidation of propylene using oxygen and hydrogen is carried out. The following procedure is employed:

A 300-cc stainless steel reactor is charged with Pd—Pb/Ti-MWW (0.7 g, Catalyst 3) or a catalyst mixture of supported noble metal catalyst (0.07 g, Catalyst 1 or Catalyst 2) and titanium zeolite (0.63 g, TS-1 or Ti-MWW powder), solvent (tert-butanol, methanol, or acetonitrile), deionized water, and modifier (amide or 0.1 M, pH=6 ammonium phosphate aqueous buffer). See Table 1 for amounts used. The reactor is then charged to 300 psig with a feed consisting of 4% hydrogen, 4% oxygen, 5% propylene, 0.5% methane and the balance nitrogen (volume %). The pressure in the reactor is maintained at 300 psig via a backpressure regulator with the feed gases passed continuously through the reactor at 1600 cc/min (measured at 23° C. and one atmosphere pressure). In order to maintain a constant solvent level in the reactor during the run, the oxygen, nitrogen and propylene feeds are passed through a two-liter stainless steel vessel (saturator) preceding the reactor, containing 1.5 liters of solvent. The reactor is stirred at 1500 rpm. The reaction mixture is heated to 60° C. and the gaseous effluent is analyzed by an online GC every hour and the liquid analyzed by offline GC at the end of the 18 hour run. The catalyst, solvent, and modifier used for each reaction run is shown in Table 1.

Propylene oxide and equivalents (“POE”), which include propylene oxide (“PO”), propylene glycol (“PG”), and propylene glycol methyl ethers (PMs), are produced during the reaction, in addition to propane formed by the hydrogenation of propylene. The results of the GC analyses are used to calculate the productivity and selectivities shown in the Table 1.

TABLE 1
Epoxidation Results

| | | Solvent | Modifier | PO/POE | Propylene | |
|---|---|---|---|---|---|---|
| Run # | Catalyst | (Amount) | (Amount) | Sel. (%)¹ | Sel. (%)² | Prod.³ |
| 2A* | 1 and | TBA-H₂O | None | 64 | 54 | 0.11 |
| | Ti-MWW | (86 g/38 g) | | | | |
| 2B* | 2 and | TBA-H₂O | Buffer | 85 | 99 | 0.46 |
| | Ti-MWW | (86 g/32 g) | (6 g) | | | |
| 2C | 2 and | TBA-H₂O | Urea | 93 | 94 | 0.44 |
| | Ti-MWW | (86 g/38 g) | (0.6 g) | | | |
| 2D | 2 and | TBA-H₂O | Formamide | 91 | 99 | 0.5 |
| | Ti-MWW | (86 g/38 g) | (1 g) | | | |
| 2E | 2 and | TBA-H₂O | Acetamide | 91 | 96 | 0.21 |
| | Ti-MWW | (86 g/38 g) | (0.6 g) | | | |
| 2F* | 2 and | Acetonitrile-H₂O | Buffer | 95 | 64 | 0.23 |
| | Ti-MWW | (86 g/32 g) | (6 g) | | | |
| 2G | 2 and | Acetonitrile-H₂O | Formamide | 96 | 86 | 0.49 |
| | Ti-MWW | (86 g/38 g) | (1 g) | | | |
| 2H* | 3 | TBA-H₂O | Buffer | 86 | 86 | 0.41 |
| | | (86 g/32 g) | (6 g) | | | |
| 2I | 3 | TBA-H₂O | Formamide | 91 | 93 | 0.42 |
| | | (86 g/38 g) | (1 g) | | | |
| 2J* | 2 and | MeOH | Buffer | 91 | 95 | 0.57 |
| | TS-1 | (100 g) | (13 g) | | | |
| 2K* | 2 and | MeOH—H₂O | Urea | 93 | 35 | 0.41 |
| | TS-1 | (100 g/13 g) | (0.6 g) | | | |

¹PO/POE Selectivity = moles PO/(moles PO + moles propylene glycols) × 100.
²Propylene Selectivity = 100 − (moles propane/moles POE + moles propane) × 100.
³Productivity = grams POE produced/gram of catalyst per hour.
*Comparative Example

We claim:

1. A process for producing an epoxide, which comprises reacting an olefin, oxygen, and hydrogen in a solvent comprising tertiary butyl alcohol or acetonitrile in the presence of an amide modifier and a catalyst comprising a titanium-MWW zeolite and a noble metal.

2. The process of claim 1 wherein the catalyst contains 0.01 to 10 weight percent of the noble metal and 0.001 to 5 weight percent of an additional metal selected from the group consisting of lead, bismuth, and rhenium.

3. The process of claim 1 wherein the noble metal is palladium.

4. The process of claim 1 wherein the noble metal is supported on the titanium-MWW zeolite.

5. The process of claim 1 wherein the noble metal is supported on a carrier.

6. The process of claim 5 wherein the carrier is selected from the group consisting of carbon, titania, zirconia, niobia, silica, alumina, silica-alumina, tantalum oxide, molybdenum oxide, tungsten oxide, titania-silica, zirconia-silica, niobia-silica, and mixtures thereof.

7. The process of claim 1 wherein the olefin is a C₂-C₆ olefin.

8. The process of claim 1 wherein the amide modifier is selected from the group consisting of urea, formamide, and acetamide.

9. The process of claim 1 wherein the solvent further comprises water.

10. A process for producing propylene oxide comprising reacting propylene, hydrogen and oxygen in a solvent comprising tertiary butyl alcohol or acetonitrile in the presence of an amide modifier and a catalyst comprising a titanium-MWW zeolite and palladium.

11. The process of claim 10 wherein the catalyst contains 0.01 to 10 weight percent of palladium and 0.001 to 5 weight percent of lead.

12. The process of claim 10 wherein the palladium is supported on the titanium-MWW zeolite.

13. The process of claim 10 wherein the noble metal is supported on a carrier selected from the group consisting of carbon, titania, zirconia, niobia, silica, alumina, silica-alumina, tantalum oxide, molybdenum oxide, tungsten oxide, titania-silica, zirconia-silica, niobia-silica, and mixtures thereof.

14. The process of claim 10 wherein the amide modifier is selected from the group consisting of urea, formamide, and acetamide.

15. The process of claim 10 wherein the solvent further comprises water.
